(19) United States
(12) Patent Application Publication
(10) Pub. No.: US 2010/0075368 A1
(43) Pub. Date: Mar. 25, 2010

(54) Media for recovery of microorganism in the presence of antibiotics

(75) Inventor: Kathleen Souza, Westboro, MA (US)

Correspondence Address: MILLIPORE CORPORATION, 290 CONCORD ROAD, BILLERICA, MA, 01821, US

(73) Assignee: Millipore Corporation, Billerica, MA (US)

(21) Appl. No.: 12/592,234

(22) Filed: Nov. 20, 2009

Related U.S. Application Data
(62) Division of application No. 11/194,714, filed on Aug. 1, 2005, now abandoned.
(60) Provisional application No. 60/601,241, filed on Aug. 13, 2004.

(51) Int. Cl. C12Q 1/04

(52) U.S. Cl. 435/34

(57) ABSTRACT

The present invention provides a media and a method of using such as media for the bioburden or sterility testing of antibiotics or environmental testing of antibiotic manufacturing areas. The medium contains one or more divalent or trivalent cation constituents, preferably of magnesium, calcium, aluminum and iron that allow for microorganism growth even in the presence of residual antibiotic.

### CROSS REFERENCE TO RELATED APPLICATIONS

This application is a Divisional patent application of U.S. application Ser. No. 11/194,714, filed on May 8, 2007, the entire contents of which are incorporated by reference herein.

### BACKGROUND

Pharmaceuticals, ophthalmics and the like need to be sterile so as to not compromise or injure the user. They need to be tested to ensure that either any microbes that are present are at accepted low levels or that no microbes exist at all before the product is released.

For most products, powder or liquid, the process is as follows:

The sample of the liquid (or of a powder dissolved into a liquid) is filtered through a microporous filter having a pore size small enough to capture any microorganisms on its surface.

The filter is then either placed on a growth medium such as an agar plate or in a medium such as a broth or a growth medium is applied to filter or an absorptive pad below filter and incubated, either at room temperature or at elevated temperatures (98° F. or so) for a period of time to allow any microorganisms to grow to a size sufficient to be enumerated and if desired identified.

The test for enumeration and identification can be visual (simply counting the number of colonies that form) or alternatively it may be done through the use of various agents to detect the presence of the microbes and to provide a signal (bio- or chemi-luminescent, radiologic, colorimetric and the like) that can be seen by the eye or through instrumentation.

One type of product that is difficult to test is antibiotics such as fluoroquinolones, aminoglycosides, tetracyclines, beta-lactams (such as penicillins, cephalosporin and others), glycopeptides, lipopeptides, macrolides, streptogramins, lincosamides, oxazolidinones, sulfonamides, polypeptide classes or antifungals such as azoles, polyenes, pyrimidine synthesis inhibitor, glucan synthesis inhibitors and chitin synthesis inhibitors.

Antibiotics are designed to kill or inhibit bacteria and other microorganisms. The current test uses a series of washes to attempt to remove all traces of the antibiotic from the filter so that microbe growth is not inhibited. This is a time consuming, costly procedure and it doesn't always work. What is needed is a better methodology for the bioburden, sterility and environmental testing of antibiotics.

### SUMMARY OF THE INVENTION

The present invention relates to a media and a method for recovering microorganisms in the presence of antibiotics. More particularly, it relates to media for bioburden and sterility testing of antibiotics. This media may also be used for environmental monitoring of microorganisms in the antibiotic manufacturing facilities.

The present invention provides a media and a method of using such for the bioburden or sterility testing of antibiotics or for testing the antibiotic manufacturing environment. The medium contains one or more divalent or trivalent cation constituents that allow for microorganism growth even in the presence of residual antibiotic.

A method for using the media is to filter an antibiotic sample through a filter having a pore size small enough to capture the suspected microorganisms and then incubate that filter on or in a growth medium that contains one or more divalent or trivalent cation constituents. The incubated filter or medium is then viewed or tested to determine the presence and if so, number and desirably, type of microorganisms present.

A second method for bioburden or sterility testing is by directly inoculating or plating an antibiotic sample in or on the medium that contains one or more divalent or trivalent cation constituents. A third method for using the media is to pull air onto a filter or directly onto or into a medium that contains one or more divalent or trivalent cations.

### DETAILED DESCRIPTION

The present invention relates to a growth media that contains one or more divalent or trivalent cation constituents within them. The presence of the one or more divalent or trivalent cation constituents allows microorganisms to grow even in the presence of residual antibiotics. It is useful for bioburden or sterility testing of antibiotics where the presence of residual antibiotics inhibits microorganism growth and provides a false negative result. It is also useful for environmental monitoring of air in an antibiotic manufacturing environment.

Any divalent or trivalent cation constituent may be used in the present invention. Preferred examples include but are not limited to magnesium, preferably in the form of magnesium sulfate and magnesium chloride; calcium preferably in the form of calcium chloride or calcium citrate; aluminum sulfate; and iron (ferrous) sulfate.

The amount of divalent or trivalent cation present in the media should be sufficient to overcome the antibiotic inhibition so as to allow any microorganisms present to grow. Generally it should be present in an amount from about 0.1 M to about 0.5M preferably from about 0.2 M to about 0.4M and more preferably about 0.3M.

Media are typically in the form of a gel, such as agar-based media or in the form of a liquid, such as broths. The media typically used in these tests are Soybean Casein Digest Broth or Agar (SCDB or SCDA), Fluid Thioglycollate Medium (FTM) and Sabouraud Dextrose Agar (SDA). Other media that are useful include but not limited to Mueller Hinton Broth or Agar, Nutrient Broth or Agar (agar may be in special cassettes or in a standard agar Petri plate).

A typical method for testing for bioburden levels or sterility of antibiotics is to filter a sample of antibiotic through a filter having a pore size small enough to trap any microorganisms and then incubate that filter in the presence of a growth medium to allow the microorganisms to grow to a size suitable for detection.

Such filters typically have a pore size of 0.45 microns or less; in some instances a pore size from about 0.1 micrometers up to 1.2 micrometers is preferred. The filters can be formed of any suitable material commonly used for such applications including but not limited to cellulose based filters such as regenerated cellulose, mixed cellulose esters, cellulose acetate, cellulose nitrate, nitro cellulose and the like, PVDF, nylons, polycarbonates and polysulfones such as polysulfone, polyethersulfone, polyarylsulfone and polyphenylsulfone.

Such filters are commercially available from a number of suppliers. Suitable filters include S-Pak™ mixed cellulose ester filters and Durapore® PVDF filters available from Millipore Corporation of Billerica, Mass.

Holders for the filters may simply be a stainless device such as a funnel or it may be a disposable, presterilized filter containing device such as a Steritest™ device, a MicropreSure™ device, Sterisure™ device, a Milliflex® filter unit or a Microfil® S device, all available from Millipore Corporation of Billerica, Mass.

Especially for sterility testing, the use of an enclosed test, such as a Steritest™ device, allows one to conduct the entire test (sampling, filtration, media addition and incubation) in a closed system. This design dramatically reduces the risks of adventitious contamination and subsequent false positives.

A typical method for testing for environmental levels of microbes in a facility is to filter a sample of air through a device having a media cassette on to which the microbes can be placed and retained and then incubate on that media to allow the microorganisms to grow to a size suitable for detection. One such system is known as the M Air T® system available from Millipore Corporation of Billerica, Mass. Also see U.S. Pat. Nos. 6,094,997 and 6,240,768. Other methods include simply leaving opened Petri dishes filled with a selected medium out in the environment to be studied and allowing falling microbes to collect on the medium's surface. The dishes are then incubated and viewed. Other methods can be and are used by those of ordinary skill in the art.

Generally, the sample after application to the media is incubated for a period of time to enable some growth of the captured microorganisms so that they can be easily detected. For traditional methods, this time can range from a minimum of 3 days for an air monitoring or bioburden sample to 14 days for the sterility test. Generally it is between about 3-14 days, more generally between about 7 and 14 days. The sample may be incubated at room temperature (around 20° C.) up to higher temperatures such as around 54° C. Typical temperature range is 20 to 35° C.

The test for enumeration and identification can be visual (simply counting the number of colonies that form) either with the naked eye or through a microscope or other magnifying device. Alternatively, it may be more complex and use various agents to detect the presence of various microbe constituents such as such as probes for DNA or RNA, agents for ATP; bioluminescence and other such well know chemical/biochemical agents to indicate the presence of these constituents and/or instruments to detect these agents to indicate the existence of and type of organisms present. For example, one well-known system incubates the microbes, lyses them and then uses reagents to detect the ATP within them. The presence of the ATP is visualized by a bioluminescent reaction of luciferine and luciferase. One such system is sold as the Microstar® system available from Millipore Corporation of Billerica, Mass. Other systems based on chromatographic indicators, fluorescent indicators, and the like are also known in the art.

### Example 1

### Direct Inoculation

Test tubes containing SCDB media in the presence or absence of magnesium cation or ciprofloxacin antibiotic were inoculated with 200 colony forming units (cfu) of S. aureus (ATCC 6538). Sample A was used with a ciprofloxacin sample (100 μg/mL) and contained no divalent cation constituent. B was used with ciprofloxacin (100 μg/mL) and contained 0.5 M magnesium cation. C was used with a control and contained 0.5M of the same divalent cation of B. D was used with a control and contained no divalent cation.

The results are shown in Table 1:

TABLE 1

| | | Divalent cation | |
|---|---|---|---|
| Antibiotic | Microorganism | added to broth | Result |
| Ciprofloxacin | S. aureus | None | No growth |
| Ciprofloxacin | S. aureus | 0.5M | Growth |
| None | S. aureus | 0.5M | Growth |
| None | S. aureus | None | Growth |

### Example 2

### Filtered

A sample of ofloxacin antibiotic (10 ml of at 4 mg/mL) was filtered through two of four Milliflex® funnels containing a Durapore® membrane. Each membrane filter was rinsed with six, 100 mL rinses of USP Fluid A, the last rinse containing approximately 20 to 60 cfu of E. coli (ATCC 8739). A control of E. coli in USP Fluid A was also filtered through a Milliflex® funnel. All membranes were then placed on growth media (Soybean Casein Digest Agar) as samples A, B, C, and D. A was used with an ofloxacin sample and contained no divalent cation constituent. B was used with ofloxacin and contained 0.5 M divalent cation (magnesium). C was used with a control and contained 0.5% divalent cation of B. D was used with a control and contained no divalent cation.

The results are shown in Table 2:

TABLE 2

| | | Divalent cation | |
|---|---|---|---|
| Antibiotic | Microorganism | added to agar | Result |
| Ofloxacin | E. coli | None | No growth |
| Ofloxacin | E. coli | 0.5M | Growth |
| None | E. coli | 0.5M | Growth |
| None | E. coli | None | Growth |

As can be seen from the examples in the presence of the antibiotics, microorganism growth was inhibited unless the media contained a divalent cation. This allows for bioburden or sterility testing of antibiotics to occur while limiting or eliminating the potential for false negatives.

### Example 3

### Sterility Testing

A sample of ofloxacin antibiotic (20 ml of at 40 mg/mL) was filtered through two of four paired Steritest™ canister sets each containing a Durapore® membrane. Each membrane filter was rinsed with three, 100 mL rinses of USP Fluid A, the last rinse containing approximately 30 cfu of B. subtilis (ATCC 6633). A control of B. subtilis in USP Fluid A was also filtered through a Steritest device. Growth media (Soybean Casein Digest Agar) A, B, C, and D was then added to each canister.

The results are shown in Table 3:

TABLE 3

| | | Divalent cation | |
|---|---|---|---|
| Antibiotic | Microorganism | added to broth | Result |
| Ofloxacin | B. subtilis | None | No growth |
| Ofloxacin | B. subtilis | 0.5M | Growth |
| None | B. subtilis | 0.5M | Growth |
| None | B. subtilis | None | Growth |

Similar results were demonstrated with ofloxacin with SCDB containing a divalent cation at concentrations of 0.1, 0.2, 0.3 and 0.4 M magnesium cation. In addition, similar results were demonstrated with two additional fluoroquinolones; moxifloxacin and ciprofloxacin with SCDB containing divalent cation (magnesium) at 0.3M concentration.

### Example 4

### Air Monitoring Testing

One method of monitoring air in an antibiotic manufacturing plant is to impact air onto an agar surface collecting both microorganisms and antibiotic on the agar surface. This test was simulated by spreading microorganisms over an agar surface containing varying amounts of magnesium cation and then placing disks containing antibiotics onto the agar surface. The zone of inhibition around each disk was measured to give an indication of the ability of the medium to neutralize the effect of the antibiotic (method followed is similar to the susceptibility disc test used in clinical microbiology). S. aureus (ATCC 6538), P. aeruginosa (ATCC 9027) or E. coli (ATCC 25922) were spread on the agar surface of SCDA containing 0.1M, 0.2M, 0.3M, 0.4M or 0.5M magnesium cation. Discs impregnated with a known amount of antibiotic were then placed onto the bacteria on the agar plate. Plates were incubated and the zone of inhibition of bacterial growth was measured. A decrease in this zone of inhibition of bacterial growth demonstrates the protective effect of the medium containing cation.

Results for three antibiotics, ofloxacin (a fluoroquinolone), streptomycin (an aminoglycoside) and doxycycline (a tetracycline), are shown in Table 4.

TABLE 4

| | | Zone of Inhibition in mm | | | | | |
|---|---|---|---|---|---|---|---|
| | | 0.0 M | | 0.2M | 0.3M | 0.4M | 0.5M |
| Antibiotic | Microorganism | cation | 0.1M cation | cation | cation | cation | cation |
| Ofloxacin | S. aureus | 23 | 11 | 8 | 6 | 7 | 6 |
| (5 ug) | | | | | | | |
| Ofloxacin | P. aeruginosa | 20 | 6 | 6 | 6 | 6 | 6 |
| (5 ug) | | | | | | | |
| Ofloxacin | E. coli | 26 | 13 | 13 | 13 | 12 | 12 |
| (5 ug) | | | | | | | |
| Streptomycin | S. aureus | 17 | 12 | Np | 7 | Np | 8 |
| (10 ug) | | | | | | | |
| Streptomycin | P. aeruginosa | 11 | 6 | Np | 6 | Np | 6 |
| (10 ug) | | | | | | | |
| Streptomycin | E. coli | 14 | 12 | Np | 9 | Np | 6 |
| (10 ug) | | | | | | | |
| Doxycycline | S. aureus | 25 | 13 | Np | 11 | Np | 13 |
| (30 ug) | | | | | | | |
| Doxycycline | P. aeruginosa | 6 | 6 | Np | 6 | Np | 6 |
| (30 ug) | | | | | | | |
| Doxycycline | E. coli | 18 | 6 | Np | 6 | Np | 6 |
| (30 ug) | | | | | | | |

Np: not performed

Similar results were observed with eight other fluoroquinolone agents, ciprofloxacin, moxifloxacin, enoxocin, enrofloxacin, levofloxacin, lemofloxacin, norfloxacin and sparfloxacin.

What is claimed:

1. A process for determining the existence of a microorganism in the presence of an antibiotic which would otherwise inhibit the growth of the microorganism and the antibiotic is not produced by the microorganism, wherein the antibiotic is selected from fluoroquinolones, tetracyclines and aminoglycosides, comprising the steps of:
a) providing a sample containing an antibiotic to be tested;
b) providing a holder;
c) providing a growth medium selected from the group consisting of gel, agar, and broth, wherein the growth medium includes one or more divalent and trivalent cation constituents of magnesium, calcium, aluminium and iron ranging from about 0.1 M to about 0.5M;
d) placing the sample in contact with the growth media;
e) incubating the sample and the growth media in the holder for a pre-selected time at a pre-selected temperature; and
f) viewing the growth media to determine the presence of any microorganisms.

2. The process of claim 1 further comprising:
d-1) providing a filter; and
d-2) filtering the sample through the filter prior to step d) and contacting the filter with the growth media before incubation.

3. The process of claim 1 further comprising:
d-3) providing a filter, and
d-4) filtering the sample through the filter prior to step d) and contacting the filter with the growth media before incubation, wherein the filter is selected from a material consisting of regenerated cellulose, mixed cellulose esters, cellulose acetate, cellulose nitrate, nitrocellulose, PVDF, nylons, polycarbonates, polysulfones, polyethersulfones, polyarylsulfones and polyphenylsulfones, and the filter has a pore size about 0.1 microns to about 1.2 microns.

4. The process of claim 1 wherein,
i) the sample is selected from a liquid and a powder dissolved in a liquid,
ii) the filter, growth media and sample in step d) are incubated for a period of time from about 0 to 14 days at a temperature from about 20° C. to about 54° C.,
iii) the viewing of the growth media to determine the presence of any microorganisms is selected from visual counting of the colony forming units, bioluminescent detection of the presence of a microbe constituent, chemiluminescent detection of the presence of a microbe constituent, or by the detection of an agent used to indicate the presence of a microbe constituent.

5. The process of claim 1 wherein the one or more cation constituents range from about 0.2M to about 0.4M.

6. The process of claim 1, wherein the medium is in the form of a gel or liquid.

7. The process of claim 1, wherein the antibiotic is selected from fluoroquinolones.

8. The process of claim 1, wherein the antibiotic is selected from tetracyclines.

9. The process of claim 1, wherein the antibiotic is selected from aminoglycosides.

10. The process of claim 1, wherein the microorganism are selected from S. aureus and P. aeruginos.

11. The process of claim 1 further comprising,
i) providing a filter,
ii) filtering the sample through the filter, and
iii) placing the filter onto the growth media before incubation,
wherein the filter is selected from the group consisting of regenerated cellulose, mixed cellulose esters, cellulose acetate, cellulose nitrate, nitrocellulose, PVDF, nylons, polycarbonates, polysulfones, polyethersulfones, polyarylsulfones and polyphenylsulfones, and the filter has a pore size less than about 0.45 microns.

12. The process of claim 1 wherein the sample is air from a manufacturing area of antibiotic manufacturing.

13. The process of claim 3 wherein the filter, growth media and sample in step d) are incubated for a period of time from about 7 to about 14 days at a temperature from about 20° C. to about 35° C.

14. The process of claim 1 wherein the one or more cation constituents range from about 0.2M to about 0.4M.

15. The process of claim 11 wherein the one or more cation constituents are in an amount about 0.3M.

16. The process of claim 1, wherein the growth medium is agar based.

17. The process of claim 1, wherein the one or more cation constituents are selected from the group consisting of magnesium sulfate, magnesium chloride, calcium chloride, calcium citrate, aluminum sulfate and iron sulfate.

18. The process of claim 1, wherein the one or more cation constituents are selected from the group consisting of magnesium and calcium.

19. The process of claim 1, wherein the medium is selected from the group consisting of Soybean Casein Digest Broth, Soybean Casein Digest Agar, Fluid Thioglycollate Medium, Sabouraud Dextrose Agar, Mueller Hinton Broth, Mueller Hinton Agar, Nutrient Broth and Nutrient Agar.

20. A process for testing the bio-burden of a sample wherein the sample includes a microorganism and an exogenously added antibiotic which would otherwise inhibit the growth of the microorganism, and the antibiotic is not produced by the microorganism, wherein the antibiotic is selected from fluoroquinolones, tetracyclines and aminoglycosides comprising the steps of:
a) providing a sample containing an antibiotic to be tested;
b) providing a holder;
c) providing a growth medium selected from the group consisting of gel, agar, and broth, wherein the growth medium includes one or more divalent and trivalent cation constituents of magnesium, calcium, aluminium and iron ranging from about 0.1 M to about 0.5M;
d) providing a filter selected from a material consisting of regenerated cellulose, mixed cellulose esters, cellulose acetate, cellulose nitrate, nitrocellulose, PVDF, nylons, polycarbonates, polysulfones, polyethersulfones, polyarylsulfones and polyphenylsulfones, and the filter has a pore size about 0.1 microns to about 1.2 microns;
e) filtering the sample through the filter;
f) contacting the filter with the growth media;
g) incubating the filter and the growth media in the holder for period of time from about 0 to 14 days at a temperature from about 20° C. to about 54° C.; and
h) viewing the growth media to determine the presence of any microorganisms.
